SEQUENCE LISTING (11 sequences)

SEQ ID NO: 1 - DNA, 2308 bp, Homo sapiens
gagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagg

SEQ ID NO: 2 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 3 - protein, 21 aa, Artificial
  synthetic peptide derived from CD25
TTDTQKSTQSVYQENLAGHCR

SEQ ID NO: 4 - protein, 20 aa, Artificial
  synthetic peptide derived from CD25
ASEESQGSRNSFPESEACPT

SEQ ID NO: 5 - protein, 20 aa, Artificial
  synthetic peptide derived from IL2-Rb
IFLETLTPDTSYELQVRVIA

SEQ ID NO: 6 - protein, 20 aa, Artificial
  synthetic peptide derived from IL-2Rb
SVDLLSLSVVCWEEKGWRRV

SEQ ID NO: 7 - protein, 20 aa, Artificial
  synthetic peptide derived from TNFR1
WKEFMRLLGLSEHEIERLEL

SEQ ID NO: 8 - protein, 20 aa, Artificial
  synthetic peptide derived from p53
MTAMEESQSDISLELPLSQE

SEQ ID NO: 9 - protein, 15 aa, Artificial
  synthetic prptide derived from HSP65
EESNTFGLQLELTEG

SEQ ID NO: 10 - DNA, 1578 bp, Rattus norvegicus
ggaccgagcccttgttctggcattctcccaggaggatgcagaaaaggggctgacccaacattctgcagagaatttcatccagttccttcctgcatcctgatcccacgtgccagggagatggagccacacttgctgatgttggggtttctctcattcaccatagtacccggctgttgggcagagctgtgtctgtatgacccaccggaggtccccaatgccacgttcaaagccctctcctacaagaacggcaccatcctaaactgtgaatgcaagagaggtttccgaagactgaatgagctggtctatatggcttgtctaggaaactcctggagcaacaactgtcagtgcacaagcaactcccatgacaactcaagagagcaagttacacctcaacctgaaggacagaaagagcaacagaccacggacacgcagaaatcaacacagtctgtgtaccaggagaaccttgcaggtcactgcagggagccccctccttggagacatgaagacaccaagagaatctaccacttcgtggaaggacagatagttctctacacgtgtattcaaggatacaaggctctacagagaggtcctgctatcagcatctgcaagacagtgtgtggggagataaggtggacgcatccccagctcacgtgtgtagatgaaaaagaacaccatcaatttctggctagtgaagaatctcaaggaagcagaaattctttcccagagagtgaggcttcctgtcccacccccaacacagacttctcacaactcacagaagcaactacaactatggagacattcgtgttcacaaaggagtatcaggtagcagtggccagctgcatcttcctgctcctcagcatcctcctcctgagtgggttcacctggcaacatagatggaggaagagcagaagaaccatctagcaagctagaacagttggagcccaagggaagatgatggactcatgaagctcaagaaacacctgaggggtcaaacgtgcactcgacgggtgcctgtctcctttcgatccctcgggtcctggaaagttatgaagtcccgagacacaatggcacatcgggaaatagcaacttcatcactaaaccgaactttccattgaagaataggatctgaccatttcagtgcagcagttctaaagctttaacgggagggagggcccaacggtgcctgtgtgttttgttttgtgtacatgtgttgatgggagctgcgatggtgtggtcacttttcgtggaacacacaatatagaaaagttgctttatgttgacttcttttggagagcccagcactaatgtaaatactccctcctgctcttccttcctcttcctcttctcttcctccttactcctcccctggtccacccacctgcacccatctacttttcttcttcctttctgttctcacaaggtcatcctaggcatcatgtatggctggctcctttctcaacctctgtttgcctaactggttctttggatttcatcacttactgatcagttttttaaaactctgggctgacaatgaggactccatgtttttagaaggaaaccccctttccactgaagctt

SEQ ID NO: 11 - DNA, 1623 bp, Mus musculus
gacacagactacacccagagaaagaagagcaagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctgaaagtcctatagtcctaagtgacgctaacctcgggtactcaccttggcaatctggatccaatgctcactggcttccttggggctaaggtaagtttcgatttcctgtcccatgtaactgcttttctgttccatatgcgctacttgagagtgtcccttgccctctttccctgcacaagccctcccatgcccagcctaacacctttccactttctttgaagagagtcttaccctgtagcccagggtggctgggagctcactatgtaggccaggttggtccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgataaaaaaaaa

(19) United States
(12) Patent Application Publication
(10) Pub. No.: US 2007/0274949 A1
(43) Pub. Date: Nov. 29, 2007

(54) Cd25 Dna Vaccines for Treating and Preventing T-Cell Mediated Diseases

(75) Inventors: Irun R. Cohen, Rehovot (IL); Avishai Mimran, Gush Etzion (IL); Francisco Quintana, Capital Federal (AR); Felix Mor, Kfar Saba (IL); Pnina Carmi, Rehovot (IL)

Correspondence Address: HESLIN ROTHENBERG FARLEY & MESITI PC, 5 COLUMBIA CIRCLE, ALBANY, NY, 12203, US

(73) Assignee: Yeda Research and Development Co. Ltd. at the Weizmann Institute of Science, Rehovot (IL)

(21) Appl. No.: 10/591,558

(22) PCT Filed:
(86) PCT No.: PCT/IL05/00273

Related U.S. Application Data
(60) Provisional application No. 60/550,308, filed on Mar. 8, 2004.

(51) Int. Cl. A61K 48/00; A61K 9/127

(52) U.S. Cl. 424/85.2; 424/93.2; 424/450; 977/907

(57) ABSTRACT

Compositions comprising nucleic acids encoding the α chain of IL-2 receptor (IL-2Ra, CD25), homologs and fragment thereof, are effective in the treatment and prevention of T cell mediated pathologies. Methods are provided for enhancing anti-ergotypic T cell activity in a subject in need thereof, and for treating or preventing T cell mediated pathologies, such as autoimmune disease, inflammatory diseases and graft rejection.

### FIELD OF THE INVENTION

The present invention is related to DNA vaccines of CD25 and fragments thereof useful in methods for the treatment of autoimmune and other T cell-mediated pathologies.

### BACKGROUND OF THE INVENTION

While the normal immune system is closely regulated, aberrations in immune response are not uncommon. In some instances, the immune system functions inappropriately and reacts to a component of the host as if it were, in fact, foreign. Such a response results in an autoimmune disease, in which the host's immune system attacks the host's own tissue. T cells, as the primary regulators of the immune system, directly or indirectly affect such autoimmune pathologies.

T cell-mediated inflammatory diseases refers to any condition in which an inappropriate T cell response is a component of the disease. This includes both diseases mediated directly by T cells, and also diseases in which an inappropriate T cell response contributes to the production of abnormal antibodies.

Numerous diseases are believed to result from autoimmune mechanisms. Prominent among these are rheumatoid arthritis, systemic lupus erythematosus, multiple sclerosis, Type I diabetes, myasthenia gravis and pemphigus vulgaris. Autoimmune diseases affect millions of individuals world-wide and the cost of these diseases, in terms of actual treatment expenditures and lost productivity, is measured in billions of dollars annually.

The existence of peripheral autoimmune T cells that recognize dominant self-antigens is a property of all healthy immune systems. The immunological dominance of self antigens such as myelin basic protein (MBP), HSP60 and insulin is associated with cellular networks consisting of the self-reacting T cells together with a network of regulatory T cells that recognize and respond to the autoimmune T cells. The two main regulatory T cells are anti-idiotypic T cells and anti-ergotypic T cells (from the Greek ergon meaning work, action).

While anti-idiotypic T cells appear to recognize the self-antigen receptors present on the pathogenic endogenous autoimmune T cells, the anti-ergotypic T cells are defined as T cells that respond to activated, syngeneic T cells independent of their idiotypic specificities. Anti-ergotypic T cells recognize as antigens the markers of the state of activation, ergotopes, of activated T cells. An example of such ergotope is the α chain of the IL-2 receptor (IL-2Ra, CD25), expression of which is up-regulated in activated T cells during T cell activation (Taniguchi and Minami, 1993; Minami et al., 1993). Anti-ergotypic T cells do not appear to respond to their target T cells in the resting state. T cell lines generated by vaccination with peptides derived from CD25 were shown to exhibit a proliferative response when cultured with activated irradiated T cells, and were suggested to be involved in protection from actively-induced EAE (Mor et al., 1996).

A comparison between the anti-ergotypic regulatory T cells and the anti-idiotypic regulatory T cells, although having some features in common, also reveals a difference in cytokine profile. While anti-idiotypic regulatory T cells secret Th1 cytokines (Cohen, 2001; Kumar et al., 2001), the anti-ergotypic regulatory T cells secrete mainly IL-10, a Th2 cytokine.

Experimental autoimmune encephalomyelitis (EAE) is a T cell mediated autoimmune disease of the central nervous system that serves as an experimental model for multiple sclerosis. Autoimmune diseases such as EAE can be prevented or treated by administering attenuated, but potentially virulent autoimmune T cells specific for the disease-related self-antigens, a procedure called T-cell vaccination (TCV). The effect of TCV was partially mediated by the in vivo activation of anti-ergotypic T cells (Lohse et al., 1989).

A preferable method for treating T cell mediated pathologies, such as autoimmune diseases, inflammatory diseases and graft rejection, includes modulating the immune system of a patient to assist the patient's natural defense mechanisms. Traditional reagents and methods used to attempt to regulate an immune response in a patient also result in unwanted side effects and have limited effectiveness. For example, immunosuppressive reagents (e.g., cyclosporin A, azathioprine, and prednisone) used to treat patients with autoimmune diseases also suppress the patient's entire immune response, thereby increasing the risk of infection. In addition, immunopharmacological reagents used to treat cancer (e.g., interleukins) are short-lived in the circulation of a patient and are ineffective except in large doses. Due to the medical importance of immune regulation and the inadequacies of existing immunopharmacological reagents, reagents and methods to regulate specific parts of the immune system have been the subject of study for many years.

Stimulation or suppression of the immune response in a patient can be an effective treatment for a wide variety of medical disorders. T lymphocytes (T cells) are one of a variety of distinct cell types involved in an immune response. The activity of T cells is regulated by antigen, presented to a T cell in the context of a major histocompatibility complex (MHC) molecule. The T cell receptor (TCR) then binds to the MHC-antigen complex. Once antigen is complexed to MHC, the MHC-antigen complex is bound by a specific TCR on a T cell, thereby altering the activity of that T cell.

WO 01/57056 of Karin discloses a method of treating rheumatoid arthritis of an individual. The method comprises the step of expressing within the individual at least an immunologically recognizable portion of a cytokine from an exogenous polynucleotide encoding the at least a portion of the cytokine, wherein a level of expression of the at least a portion of the cytokine is sufficient to induce the formation of anti-cytokine immunoglobulins which serve for neutralizing or ameliorating the activity of a respective and/or cross reactive endogenous cytokine, to thereby treat rheumatoid arthritis. U.S. Pat. No. 6,316,420 to Karin and coworkers further discloses DNA cytokine vaccines and use of same for protective immunity against multiple sclerosis. WO 02/16549 of Cohen Irun relates to DNA vaccines useful for the prevention and treatment of ongoing autoimmune diseases. The compositions and methods of the invention feature the CpG oligonucleotide, preferably in a motif flanked by two 5′ purines and two 3′ pyrimidines. The vaccine may further comprise DNA encoding a specific antigen, or the peptide antigen itself.

WO 00/27870 of Naparstek and colleagues discloses a series of related peptides derived from heat shock proteins Hsp65 and Hsp60, their sequences, antibodies, and use as vaccines for conferring immunity against autoimmune and/or inflammatory disorders such as arthritis. These peptides are intended by the inventors to represent the shortest sequence or epitope that is involved in protection of susceptible rat strains against adjuvant induced arthritis. These sequences further disclose what the inventors identify as the common “protective motif”.

At present, there are no effective treatments for T-cell mediated autoimmune diseases. Usually, only the symptoms can be treated, while the disease continues to progress, often resulting in severe debilitation or death. Thus, there exists a long-felt need for an effective means of curing or ameliorating T cell mediated pathologies. Such a treatment should ideally control the inappropriate T cell response, rather than merely reducing the symptoms. Nowhere in the background art is it taught or suggested that DNA vaccines comprising polynucleotides encoding CD25 may be used specifically to prevent or treat T-cell mediated autoimmune diseases.

### SUMMARY OF THE INVENTION

The present invention provides compositions comprising nucleic acid molecules encoding the α chain of IL-2 receptor (IL-2Ra, CD25), homologs and fragments thereof, effective in the treatment and prevention of T cell mediated pathologies. The invention further provides methods for enhancing anti-ergotypic T cell activity in a subject in need thereof, and for treating or preventing T cell mediated pathologies, such as autoimmune diseases, inflammatory diseases and graft rejection.

DNA vaccination represents a novel means of expressing antigen in vivo for the generation of both humoral and cellular immune responses. The present invention is based in part on the unexpected discovery that DNA vaccination with CD25 elicits protective immunity against T cell mediated pathologies such as autoimmune diseases, as exemplified by the animal disease model of adjuvant arthritis (AA), a T cell mediated autoimmune disease that serves as an experimental model for rheumatoid arthritis.

According to the present invention it is now disclosed that it is possible to treat or prevent T cell-mediated pathologies by using DNA vaccines encoding CD25, fragments and analogs derivatives thereof.

According to the present invention, expression of nucleic acid molecules encoding CD25, which results in systemic or localized production of an effective amount of CD25, elicits anti-ergotypic T cell responses.

Without wishing to be bound by any theory or mechanism of action, the anti-ergotypic T cell response is characterized by a reduction in the secretion of IFNγ and an increase in the secretion of IL-10 in said T cells.

The use of DNA vaccination for the generation of cellular immune responses is particularly advantageous. It provides an effective therapeutic composition that enables the safe treatment of a subject with potentially toxic proteins. The nucleic acid-based therapeutic compositions of the present invention can provide long-term expression of CD25. Such long-term expression allows for the maintenance of an effective, but non-toxic, dose of the encoded protein to treat a disease and limits the frequency of administration of the therapeutic composition needed to treat a subject. In addition, because of the lack of toxicity, these therapeutic compositions can be used in repeated treatments.

In one aspect, the invention provides a DNA vaccine composition comprising a recombinant construct comprising an isolated nucleic acid sequence encoding an antigen selected from CD25, homologs and fragments thereof; the nucleic acid sequence being operably linked to one or more transcription control sequences; and a pharmaceutically acceptable carrier, adjuvant, excipient or diluent.

In one embodiment, the isolated nucleic acid sequence comprises the coding sequence of human CD25. In a preferred embodiment, the nucleic acid molecule comprises a nucleic acid sequence as set forth in SEQ ID NO:1 (gi:4557666). In another preferred embodiment, the isolated nucleic acid sequence encodes a polypeptide having an amino acid sequence as set forth in SEQ ID NO:2 (gi:4557667). In other preferred embodiments, the isolated nucleic acid sequence encodes a CD25 fragment having an amino acid sequence as set forth in any one of SEQ ID NOS:3 and 4 (see Examples below).

The compositions of the present invention are useful for the treatment and prevention of T cell-mediated pathologies in a subject in need thereof, including, but not limited to, autoimmune diseases, graft rejection and T cell mediated inflammatory diseases.

In certain embodiments, the T cell-mediated autoimmune diseases include, but are not limited to, multiple sclerosis, rheumatoid arthritis, autoimmune neuritis, systemic lupus erythematosus, psoriasis, Type I diabetes mellitus, Sjogren's disease, thyroid disease and myasthenia gravis.

In other embodiments the subject in need thereof is selected from the group consisting of humans and non-human mammals. In a preferred embodiment, the subject is human.

In another aspect, the invention provides a method of preventing or inhibiting the development of a T-cell mediated pathology, comprising administering to a subject in need thereof a therapeutically effective amount of a pharmaceutical composition comprising: (a) a recombinant construct, said recombinant construct comprising an isolated nucleic acid sequence encoding an antigen selected from: CD25, homologs and fragments thereof, wherein the nucleic acid sequence is operably linked to one or more transcription control sequences; and (b) a pharmaceutically acceptable carrier, excipient or diluent.

In one embodiment, the isolated nucleic acid sequence comprises the coding sequence of human CD25. In a preferred embodiment, the nucleic acid molecule comprises a nucleic acid sequence as set forth in SEQ ID NO:1. In other preferred embodiments, the isolated nucleic acid sequence encodes a polypeptide or peptide having an amino acid sequence as set forth in any one of SEQ ID NOS:2-4. In another embodiment, the antigen is expressed in sufficient amount and duration to increase anti-ergotypic T cell response in said subject, thereby inhibiting the development of said T-cell mediated pathology.

In another aspect, the invention provides a method for preventing or inhibiting the development of a T-cell mediated pathology comprising the steps of (a) obtaining cells from a subject; (b) transfecting the cells in vitro with a recombinant construct comprising an isolated nucleic acid sequence encoding an antigen selected from: CD25, homologs and fragments thereof, the nucleic acid sequence being operably linked to one or more transcription control sequences; and (c) reintroducing a therapeutically effective number of the transfected cells to the subject, thereby preventing or inhibiting the development of the T-cell mediated pathology.

In one embodiment, the isolated nucleic acid sequence comprises the coding sequence of human CD25. In a preferred embodiment, the nucleic acid molecule comprises a nucleic acid sequence as set forth in SEQ ID NO:1. In other preferred embodiments, the isolated nucleic acid sequence encodes a polypeptide or peptide having an amino acid sequence as set forth in any one of SEQ ID NOS:2-4. In another embodiment, the antigen is expressed in sufficient amount and duration to increase anti-ergotypic T cell response in said subject, thereby inhibiting the development of said T-cell mediated pathology.

### BRIEF DESCRIPTION OF THE DRAWINGS

FIG. 1 demonstrates that the anti-ergotypic T cell response in naïve rats is down regulated by AA induction. LN cells from (A) naïve rats (Mean Spontaneous Proliferation [MSP]=153 cpm) or (B) DLN cells from rats at day 22 of AA induction (MSP=159 cpm) were pooled from 3 rats, and the T cell response to activated (A6-S) or resting (A6-R) T cells at different stimulator cell concentrations was measured. Proliferative responses are presented as the stimulation index (SI)±SEM of quadruplicate cultures. This is a representative experiment of three repetitions.

FIG. 2 demonstrates that CD25 DNA vaccination protects against AA. (A) Groups of 8 rats each were untreated, vaccinated with the empty vector (pcDNA3), the CD25 gene, or the CD132 gene, prior to AA induction (day 0). AA scores were assessed every day or two starting at day 11. The mean ±SEM disease score is shown. Scores of the CD25 vaccinated group were significantly reduced compared to the pcDNA3 group for each of the days 14-26 (p<0.01). The p value of day 26 is indicated. (B) Ankle swelling measured at day 26 after AA induction. The results are presented in millimeters, mean ±SEM, measured for the hind limb ankle diameter. The p value compares the CD25 and pcDNA3 groups.

FIG. 3 demonstrates the IgG responses to ergotope peptides following DNA vaccination. Sera of rats vaccinated with (A) an empty vector, (B) CD132 or (C) CD25, were obtained 10 days after the 3 DNA vaccination and analyzed for IgG antibodies to immunogenic peptides of different ergotopes. Each group was of 8 rats. Single rats are represented by circles. The group average is represented by the grid. * indicates p<0.001 compared to both control groups and also to the b1/b2 peptides of the same group.

FIG. 4 demonstrates the T cell responses to IL-2R α and β-chain peptides after AA induction. DLN cells from each of the four groups, non-treated (MSP=159 cpm), pcDNA3 (MSP=198 cpm), CD25 (MSP=223 cpm) and CD132 vaccinated (MSP=305 cpm) were pooled from 3 rats, and their anti-ergotypic responses were measured on day 22 after AA induction. Two α-chain (a1, a2) and two β-chain (b1, b2) peptides were used as ergotopes. A control peptide from the p53 protein (p53-1) was included. Proliferative responses are presented as the stimulation index (SI)±SEM of quadruplicate cultures. * indicates p<0.02, compared to the non-protected CD132 vaccinated group, for all the four peptides. This is a representative experiment of three repetitions.

FIG. 5 demonstrates the anti-ergotypic T-cell proliferative response following DNA vaccination. Ten days after DNA vaccination, DLN cells of 3 rats per group were pooled from (A) empty vector vaccinated rats (MSP=229 cpm) or (B) from CD25 vaccinated rats (MSP=164 cpm), and the T-cell responses to activated (A6-S) or resting (A6-R) irradiated T cells were measured. The test was repeated at day 22 after AA induction in (C) rats vaccinated with the empty vector (MSP=198 cpm) or (D) with the CD25 gene (MSP=223 cpm). Stimulator cells were used at the indicated doses. Proliferative responses are presented as the stimulation index (SI)±SEM of quadruplicate cultures. This is a representative experiment of three repetitions.

FIG. 6 demonstrates the cytokine secretion by anti-ergotypic T cells. (A-B) The media of the DLN cells of the three groups, non-treated, pcDNA3 and CD25 vaccinated, responding to activated (A6-S) or resting (A6-R) T cells at day 22 of AA induction were taken after 72 hours in culture and analyzed by ELISA for (A) IFNγ or (B) IL-10. The results are presented in pg/ml. This is a representative experiment of three repetitions. The p values indicate a significant decrease in IFNγ secretion and an increase in IL-10 secretion, compared to rats vaccinated with the empty vector. (C-D) The media of the DLN cells of the three groups, non-treated, pcDNA3 and CD25 vaccinated, and responding to α or β peptides at day 22 to AA induction were taken after 72 hours in culture and analyzed by ELISA for (C) IFNγ or (D) IL-10 secretion. The results are presented in pg/ml. This is a representative experiment of three repetitions.

FIG. 7 demonstrates the T cell proliferation in response to AA antigens. DLN cells from each of the three groups, non-treated (MSP=159 cpm), pcDNA3 (MSP=198 cpm) and CD25 vaccinated (MSP=223 cpm), were pooled from 3 rats and their responses were measured on day 22 after AA induction. Stimulating antigens were PPD or the p180 peptide of Mt HSP65. Proliferative responses are presented as the stimulation index (SI)±SEM of quadruplicate cultures. Proliferation to PPD of DLN cells from CD25 vaccinated rats was significantly higher (p=0.003) than that of pcDNA3 vaccinated rats. This is a representative experiment of three repetitions.

FIG. 8 demonstrates the cytokine secretion by DLN cells proliferating to AA antigens. The media of the proliferating DLN cells of the three groups, non-treated, pcDNA3 and CD25 vaccinated, responding to PPD or p180, were taken after 72 hours in culture and analyzed by ELISA for (A) IFNγ, (B) TNFα or (C) IL-10. The results are presented as pg/ml. This is a representative experiment of three repetitions. * indicates the p value in comparison with pcDNA3 vaccinated group; # indicates the p value in comparison with the non-treated group.

### DETAILED DESCRIPTION OF THE INVENTION

The present invention relates to a novel product and process for controlling regulatory T cell activity. It is now known for the first time that a composition comprising a nucleic acid molecules encoding the a chain of the IL-2 receptor (IL-2Ra, CD25), fragments and homologs thereof, is an effective therapeutic reagent for treating T cell-mediated diseases.

The present invention is based in part on studies of the role of the immune response to CD25 in adjuvant-induced arthritis (AA) in experimental rats, using DNA vaccines encoding CD25. Surprisingly, it was discovered that DNA vaccination with constructs encoding CD25 protected the rats from AA and led to a shift in the cytokine profile of T cells responding to disease target antigens from Th1 to Th2. The protection was found to be associated with the induction of an anti-ergotypic response with CD25 epitopes as ergotopes.

The term “anti-ergotypic T cell response” refers to the activation of regulatory anti-ergotypic T cells. In various embodiments, the anti-ergotypic T cell response may be measured as increased T cell proliferation response to activated syngeneic T cells. Alternatively, the activation of regulatory anti-ergotypic T cells may be determined by measuring the secretion level of cytokines by said T cells.

It is demonstrated herein that protection from AA induced by CD25 DNA vaccination is associated with a shift in the cytokine phenotype from IFNγ and TNFα towards IL-10, thereby driving the differentiation of activated T cells from a Th1-like to a Th2-like phenotype in both the anti-ergotypic response and response to the antigens targeted in the disease. Without wishing to be bound by any theory or mechanism of action, preventing or ameliorating of T cell-mediated pathology by treatment with CD25 and DNA vaccination with constructs expressing CD25 might be related to a shift in the cytokines secreted by the responding T cells. In that respect, the cytokine balance between the anti-ergotypic T cells and the effector T cells (e.g. autoimmune T cells) may affect the whole cytokine environment. In a disease state, the activated T cells causing the disease seem to be the ones controlling the cytokine environment by secreting mainly Th1 cytokines, IFNγ and TNFα. These Th1 cytokines might also have an inhibitory effect on the activation of the anti-ergotypic T cells, which in this state secrete IFNγ, and do not proliferate. The compositions and methods of the invention may boost the anti-ergotypic T cells, leading to their preservation and secretion of IL-10. The IL-10 could help drive the differentiation of the otherwise pathogenic T cells towards a Th2 phenotype.

### T Cell Mediated Pathologies

In one aspect, the present invention provides methods for treating or preventing a T cell mediated pathology. The term “T-cell mediated pathology” refers to any condition in which an inappropriate T cell response is a component of the pathology. The term is intended to include both diseases directly mediated by T cells, and also diseases in which an inappropriate T cell response contributes to the production of abnormal antibodies, as well as graft rejection.

In one embodiment of the invention, the composition is useful for treating a T cell-mediated autoimmune disease, including but not limited to: multiple sclerosis, rheumatoid arthritis, autoimmune neuritis, systemic lupus erythematosus (SLE), psoriasis, Type I diabetes mellitus, Sjogren's disease, thyroid disease, myasthenia gravis, sarcoidosis, autoimmune uveitis, inflammatory bowel disease (Crohn's and ulcerative colitis) and autoimmune hepatitis.

In other embodiments the composition is useful for treating a Th1-associated inflammatory disease, e.g. delayed-type hypersensitivity responses (DTH) and Th1 mediated allergic responses which result in skin sensitivity and inflammation, such as contact dermatitis.

In other embodiments, the composition is useful for treating graft rejection, including allograft rejection or graft-versus-host disease.

### DNA Vaccines and Related Methods

The present invention provides an effective method of DNA vaccination for T cell mediated autoimmune diseases, which avoids many of the problems associated with other methods of treatment. By vaccinating, rather than passively administering heterologous antibodies, the host's own immune system is mobilized to suppress the autoaggressive T cells. Thus, the suppression is persistent and may involve any and all immunological mechanisms in effecting that suppression. This multi-faceted response is more effective than the uni-dimensional suppression achieved by passive administration of monoclonal antibodies or extant-derived regulatory T cell clones.

The present invention relates to the use of a recombinant construct, said recombinant construct comprising an isolated nucleic acid sequence encoding CD25, or a fragment thereof, in order to elicit anti-ergotypic T cell response. Such response is required for example in T cell mediated autoimmune diseases in which the balance between the anti-ergotypic T cells and the autoimmune T cells is disturbed. In one embodiment, said nucleic acid sequence is the coding sequence encoding the α chain of the IL-2 receptor, or a fragment thereof.

The isolated nucleic acid sequence encoding CD25 may include DNA, RNA, or derivatives of either DNA or RNA. An isolated nucleic acid sequence encoding CD25 can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional CD25 of the present invention.

A nucleic acid molecule homolog can be produced using a number of methods known to those skilled in the art (see, for example, Sambrook et al., 1989). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologs can be selected from a mixture of modified nucleic acids by screening for the function of the protein encoded by the nucleic acid with respect to the induction of an anti-ergotypic response, for example by the methods described herein.

One embodiment of the present invention is an isolated CD25-encoding nucleic acid sequence that encodes at least a portion of a full-length CD25, or a homolog of CD25. As used herein, “at least a portion of CD25” refers to a portion of CD25 protein capable of increasing the anti-ergotypic T cell response. In certain embodiments, the portion of CD25 protein comprises one or more MHC II binding motifs. It is well-established in the art that class II MHC molecules bind to peptides 12-15 amino acid residues in length, with a minimum length perhaps as short as 7-9 amino acid residues. Thus, the CD25 fragments encoded by the nucleic acid molecules of the invention are preferably at least about 7-9 amino acids in length and comprise MHC II binding motifs. The identification of suitable CD25 fragments comprising MHC II binding motifs is within the abilities of those of skill in the art (see, for example, Reizis et al., 1996).

In another preferred embodiment, a CD25 nucleic acid sequence of the present invention encodes an entire coding region of CD25. As used herein, a homolog of CD25 is a protein having an amino acid sequence that is sufficiently similar to a natural CD25 amino acid sequence that a nucleic acid sequence encoding the homolog encodes a protein capable of increasing the anti-ergotypic T cell response.

In one aspect, the nucleic acid molecule encodes human CD25. In a preferred embodiment, the nucleic acid molecule comprises a nucleic acid sequence as set forth in SEQ ID NO:1 (gi:4557666). In another preferred embodiment, the isolated nucleic acid sequence encodes a polypeptide having an amino acid sequence as set forth in SEQ ID NO:2 (gi:4557667). In other preferred embodiments, the isolated nucleic acid sequence encodes a CD25 fragment having an amino acid sequence as set forth in any one of SEQ ID NOS:3 and 4 (see Examples below).

A polynucleotide or oligonucleotide sequence can be deduced from the genetic code of a protein, however, the degeneracy of the code must be taken into account. Nucleic acid sequences of the invention include sequences, which are degenerate as a result of the genetic code, which sequences may be readily determined by those of ordinary skill in the art.

The oligonucleotides or polynucleotides of the invention may contain a modified internucleoside phosphate backbone to improve the bioavailability and hybridization properties of the oligonucleotide or polynucleotide. Linkages are selected from the group consisting of phosphodiester, phosphotriester, methylphosphonate, phosphoroselenoate, phosphorodiselenoate, phosphoroanilothioate, phosphoroanilidate, phosphoramidate, phosphorothioate, phosphorodithioate or combinations thereof.

Additional nuclease linkages include alkylphosphotriester such as methyl- and ethylphosphotriester, carbonate such as carboxymethyl ester, carbamate, morpholino carbamate, 3′-thioformacetal, silyl such as dialkyl(C₁-C₆)— or diphenylsilyl, sulfamate ester, and the like. Such linkages and methods for introducing them into oligonucleotides are described in many references, e.g. reviewed generally by Peyman and Ulmann, Chemical Reviews, 90:1543-584 (1990).

The present invention includes a nucleic acid sequence of the present invention operably linked to one or more transcription control sequences to form a recombinant molecule. The phrase “operably linked” refers to linking a nucleic acid sequence to a transcription control sequence in a manner such that the molecule is able to be expressed when transfected (i.e., transformed, transduced or transfected) into a host cell. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in animal, bacteria, helminth, insect cells, and preferably in animal cells. More preferred transcription control sequences include, but are not limited to RSV control sequences, CMV control sequences, retroviral LTR sequences, SV-40 control sequences and β-actin control sequences as well as other sequences capable of controlling gene expression in eukaryotic cells. Additional suitable transcription control sequences include tissue-specific promoters and enhancers (e.g., T cell-specific enhancers and promoters). Transcription control sequences of the present invention can also include naturally occurring transcription control sequences naturally associated with a gene encoding CD25 of the present invention.

According to still further features in the described preferred embodiments the recombinant construct is a eukaryotic expression vector.

According to still further features in the described preferred embodiments the expression vector is selected from the group consisting of pcDNA3, pcDNA3.1 (+/−), pZeoSV2(+/−), pSecTag2, pDisplay, pEF/myc/cyto, pCMV/myc/cyto, pCR3.1, pCI, pBK-RSV, pBK-CMV, pTRES and their derivatives.

According to the present invention, a host cell can be transfected in vivo (i.e., in an animal) or in vitro (i.e., outside of an animal, such as in tissue culture). Transfection of a nucleic acid molecule into a host cell can be accomplished by any method by which a nucleic acid molecule can be inserted into the cell. Transfection techniques include, but are not limited to, transfection, electroporation, microinjection, lipofection, adsorption, and protoplast fusion. Preferred methods to transfect host cells in vivo include lipofection and adsorption.

A recombinant cell of the present invention comprises a cell transfected with a nucleic acid molecule that encodes CD25 or an analog or fragment thereof.

It may be appreciated by one skilled in the art that use of recombinant DNA technologies can improve expression of transfected nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operably linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgamo sequences), modification of nucleic acid molecules of the present invention to correspond to the codon usage of the host cell, and deletion of sequences that destabilize transcripts. The activity of an expressed recombinant protein of the present invention may be improved by fragmenting, modifying, or derivatizing nucleic acid molecules encoding such a protein.

According to yet another aspect of the present invention there is provided a pharmaceutical composition suitable for effecting the above methods of the present invention. In one embodiment, the composition is a DNA vaccine composition comprising a recombinant construct comprising an isolated nucleic acid sequence encoding an antigen selected from CD25, homologs and fragments thereof; the nucleic acid sequence being operably linked to one or more transcription control sequences; and a pharmaceutically acceptable carrier, adjuvant, excipient or diluent.

In one embodiment of the invention, the composition is useful for treating or preventing the development of a T cell-mediated pathology in a subject in need thereof, as described herein. In another embodiment, the composition is useful for enhancing anti-ergotypic T cell activity in a subject in need thereof.

The pharmaceutical composition of the invention is administered to a subject in need of said treatment in a therapeutically effective amount. According to the present invention, a “therapeutically effective amount” is an amount that when administered to a patient is sufficient to inhibit, preferably to eradicate, a T cell mediated pathology. According certain embodiments, the subject is selected from the group consisting of humans, dogs, cats, sheep, cattle, horses and pigs. In a preferred embodiment, the subject is human.

In another embodiment of the present invention, a therapeutic composition further comprises a pharmaceutically acceptable carrier. As used herein, a “carrier” refers to any substance suitable as a vehicle for delivering a nucleic acid molecule of the present invention to a suitable in vivo or in vitro site. As such, carriers can act as a pharmaceutically acceptable excipient of a therapeutic composition containing a nucleic acid molecule of the present invention. Preferred carriers are capable of maintaining a nucleic acid molecule of the present invention in a form that, upon arrival of the nucleic acid molecule to a cell, the nucleic acid molecule is capable of entering the cell and being expressed by the cell. Carriers of the present invention include: (1) excipients or formularies that transport, but do not specifically target a nucleic acid molecule to a cell (referred to herein as non-targeting carriers); and (2) excipients or formularies that deliver a nucleic acid molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- and o-cresol, formalin and benzol alcohol. Preferred auxiliary substances for aerosol delivery include surfactant substances non-toxic to a subject, for example, esters or partial esters of fatty acids containing from about six to about twenty-two carbon atoms. Examples of esters include, caproic, octanoic, lauric, palmitic, stearic, linoleic, linolenic, olesteric, and oleic acids. Other carriers can include metal particles (e.g., gold particles) for use with, for example, a biolistic gun through the skin. Therapeutic compositions of the present invention can be sterilized by conventional methods.

Targeting carriers are herein referred to as “delivery vehicles.” Delivery vehicles of the present invention are capable of delivering a therapeutic composition of the present invention to a target site in a subject. A “target site” refers to a site in a subject to which one desires to deliver a therapeutic composition. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a target cell can be introduced to the outer surface of a liposome delivery vehicle so as to target the delivery vehicle to the target cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

A preferred delivery vehicle of the present invention is a liposome. A liposome is capable of remaining stable in a subject for a sufficient amount of time to deliver a nucleic acid molecule of the present invention to a preferred site in the subject. A liposome of the present invention is preferably stable in the subject into which it has been administered for at least about 30 minutes, more preferably for at least about 1 hour and even more preferably for at least about 24 hours.

A liposome of the present invention comprises a lipid composition that is capable of targeting a nucleic acid molecule of the present invention to a particular, or selected, site in a subject. Preferably, the lipid composition of the liposome is capable of targeting to any organ of a subject, more preferably to the lung, liver, spleen, heart brain, lymph nodes and skin of a subject.

A liposome of the present invention comprises a lipid composition that is capable of fusing with the plasma membrane of the targeted cell to deliver a nucleic acid molecule into a cell. Preferably, the transfection efficiency of a liposome of the present invention is about 0.5 microgram (μg) of DNA per 16 nanomole (nmol) of liposome delivered to about 10⁶ cells, more preferably about 1.0 μg of DNA per 16 nmol of liposome delivered to about 10⁶ cells, and even more preferably about 2.0 μg of DNA per 16 nmol of liposome delivered to about 10⁶ cells.

A preferred liposome of the present invention is between about 100 and 500 nanometers (nm), more preferably between about 150 and 450 nm and even more preferably between about 200 and 400 nm in diameter.

Suitable liposomes for use with the present invention include any liposome. Preferred liposomes of the present invention include those liposomes standardly used in, for example, gene delivery methods known to those of skill in the art. More preferred liposomes comprise liposomes having a polycationic lipid composition and/or liposomes having a cholesterol backbone conjugated to polyethylene glycol.

Complexing a liposome with a nucleic acid molecule of the present invention can be achieved using methods standard in the art. A suitable concentration of a nucleic acid molecule of the present invention to add to a liposome includes a concentration effective for delivering a sufficient amount of nucleic acid molecule to a cell such that the cell can produce sufficient CD25 protein to regulate effector cell immunity in a desired manner. Preferably, from about 0.1 μg to about 10 μg of nucleic acid molecule of the present invention is combined with about 8 nmol liposomes, more preferably from about 0.5 μg to about 5 μg of nucleic acid molecule is combined with about 8 nmol liposomes, and even more preferably about 1.0 μg of nucleic acid molecule is combined with about 8 nmol liposomes.

Another preferred delivery vehicle comprises a recombinant virus particle vaccine. A recombinant virus particle vaccine of the present invention includes a therapeutic composition of the present invention, in which the recombinant molecules contained in the composition are packaged in a viral coat that allows entrance of DNA into a cell so that the DNA is expressed in the cell. A number of recombinant virus particles can be used, including, but not limited to, those based on alphaviruses, poxviruses, adenoviruses, herpesviruses, arena virus and retroviruses.

Another preferred delivery vehicle comprises a recombinant cell vaccine. Preferred recombinant cell vaccines of the present invention include cell vaccines, in which allogeneic (i.e., cells derived from a source other than a patient, but that are histiotype compatible with the patient) or autologous (i.e., cells isolated from a patient) cells are transfected with recombinant molecules contained in a therapeutic composition, irradiated and administered to a patient by, for example, intradennal, intravenous or subcutaneous injection. Therapeutic compositions to be administered by cell vaccine, include recombinant molecules of the present invention without carrier.

In order to treat a subject with disease, a therapeutic composition of the present invention is administered to the subject in an effective manner such that the composition is capable of treating that subject from disease. For example, a recombinant molecule, when administered to a subject in an effective manner, is able to stimulate effector cell immunity in a manner that is sufficient to alleviate the disease afflicting the subject. According to the present invention, treatment of a disease refers to alleviating a disease and/or preventing the development of a secondary disease resulting from the occurrence of a primary disease. An effective administration protocol (i.e., administering a therapeutic composition in an effective manner) comprises suitable dose parameters and modes of administration that result in treatment of a disease. Effective dose parameters and modes of administration can be determined using methods standard in the art for a particular disease. Such methods include, for example, determination of survival rates, side effects (i.e., toxicity) and progression or regression of disease.

In accordance with the present invention, a suitable single dose size is a dose that is capable of treating a subject with disease when administered one or more times over a suitable time period. Doses can vary depending upon the disease being treated. Doses of a therapeutic composition of the present invention suitable for use with direct injection techniques can be used by one of skill in the art to determine appropriate single dose sizes for systemic administration based on the size of a subject. A suitable single dose of a therapeutic composition to treat a T-cell mediated pathology is a sufficient amount of CD25-encoding recombinant sequence to reduce, and preferably eliminate, the T-cell mediated pathology following transfection of the recombinant molecules into cells. A preferred single dose of CD25-encoding recombinant molecule is an amount that, when transfected into a target cell population leads to the production of from about 250 femtograms (fg) to about 1 μg, preferably from about 500 fg to about 500 picogram (pg), and more preferably from about 1 pg to about 100 pg of CD25 per transfected cell.

A preferred single dose of CD25-encoding recombinant molecule complexed with liposomes, is from about 100 μg of total DNA per 800 nmol of liposome to about 2 mg of total recombinant molecules per 16 micromole (μmol) of liposome, more preferably from about 150 μg per 1.2 μmol of liposome to about 1 mg of total recombinant molecules per 8 μmol of liposome, and even more preferably from about 200 μg per 2 μmol of liposome to about 400 μg of total recombinant molecules per 3.2 μmol of liposome.

A preferred single dose of CD25-encoding recombinant molecule in a non-targeting carrier to administer to a subject, is from about 12.5 μg to about 20 mg of total recombinant molecules per kg body weight, more preferably from about 25 μg to about 10 mg of total recombinant molecules per kg body weight, and even more preferably from about 125 μg to about 2 mg of total recombinant molecules per kg body weight.

It will be obvious to one of skill in the art that the number of doses administered to a subject is dependent upon the extent of the disease and the response of an individual patient to the treatment. Thus, it is within the scope of the present invention that a suitable number of doses includes any number required to cause regression of a disease. A preferred protocol is monthly administrations of single doses (as described above) for up to about 1 year. A preferred number of doses of a therapeutic composition comprising CD25-encoding recombinant molecule in a non-targeting carrier or complexed with liposomes is from about 1 to about 10 administrations per patient, preferably from about 2 to about 8 administrations per patient, and even more preferably from about 3 to about 5 administrations per person. Preferably, such administrations are given once every 2 weeks until signs of remission appear, then once a month until the disease is gone.

A therapeutic composition is administered to a subject in a fashion to enable expression of the administered recombinant molecule of the present invention into a curative protein in the subject to be treated for disease. A therapeutic composition can be administered to a subject in a variety of methods including, but not limited to, local administration of the composition into a site in a subject, and systemic administration.

Therapeutic compositions to be delivered by local administration include: (a) recombinant molecules of the present invention in a non-targeting carrier (e.g., as “naked” DNA molecules, such as is taught, for example in Wolff et al., 1990); and (b) recombinant molecules of the present invention complexed to a delivery vehicle of the present invention. Suitable delivery vehicles for local administration comprise liposomes. Delivery vehicles for local administration can further comprise ligands for targeting the vehicle to a particular site.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site. Systemic administration is particularly advantageous when organs, in particular difficult to reach organs (e.g., heart, spleen, lung or liver) are the targeted sites of treatment.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., 1992, which is incorporated herein by reference in its entirety). Oral delivery can be performed by complexing a therapeutic composition of the present invention to a carrier capable of withstanding degradation by digestive enzymes in the gut of a subject. Examples of such carriers, include plastic capsules or tablets, such as those known in the art. Topical delivery can be performed by mixing a therapeutic composition of the present invention with a lipophilic reagent (e.g., DMSO) that is capable of passing into the skin.

Suitable embodiments, single dose sizes, number of doses and modes of administration of a therapeutic composition of the present invention useful in a treatment method of the present invention are disclosed in detail herein.

A therapeutic composition of the present invention is also advantageous for the treatment of autoimmune diseases in that the composition suppresses the harmful stimulation of T cells by autoantigens (i.e., a “self”, rather than a foreign antigen). CD25-encoding recombinant molecules in a therapeutic composition, upon transfection into a cell, produce CD25 or a fragment or homolog thereof that reduces the harmful activity of T cells involved in an autoimmune disease. A preferred therapeutic composition for use in the treatment of autoimmune disease comprises CD25-encoding recombinant molecule of the present invention or a fragment thereof. A more preferred therapeutic composition for use in the treatment of autoimmune disease comprises a recombinant molecule encoding CD25 or a homolog or fragment thereof combined with a non-targeting carrier of the present invention, preferably saline or phosphate buffered saline.

Such a therapeutic composition of the present invention is particularly useful for the treatment of autoimmune diseases, including but not limited to: multiple sclerosis, rheumatoid arthritis, autoimmune neuritis, systemic lupus erythematosus (SLE), psoriasis, Type I diabetes mellitus, Sjogren's disease, thyroid disease, myasthenia gravis, sarcoidosis, autoimmune uveitis, inflammatory bowel disease (Crohn's and ulcerative colitis) and autoimmune hepatitis.

A preferred single dose of nucleic acid molecule encoding CD25 or a fragment or homolog thereof in a non-targeting carrier to administer to a subject to treat an autoimmune disease is from about 12.5 μg to about 20 mg of total recombinant molecules per kg body weight, more preferably from about 25 μg to about 10 mg of total recombinant molecules per kg body weight, and even more preferably from about 125 μg to about 2 mg of total recombinant molecules per kg body weight.

The number of doses of CD25-encoding recombinant molecule in a non-targeting carrier to be administered to a subject to treat an autoimmune disease is an injection about once every 6 months, more preferably about once every 3 months, and even more preferably about once a month.

A preferred method to administer a therapeutic composition of the present invention to treat an autoimmune disease is by local administration, preferably direct injection. Direct injection techniques are particularly important in the treatment of an autoimmune disease. Preferably, a therapeutic composition is injected directly into muscle cells in a patient, which results in prolonged expression (e.g., weeks to months) of a recombinant molecule of the present invention. Preferably, a recombinant molecule of the present invention in the form of “naked DNA” is administered by direct injection into muscle cells in a patient.

Methods of treating a disease according to the invention may include administration of the pharmaceutical compositions of the present invention as a single active agent, or in combination with additional methods of treatment. The methods of treatment of the invention may be in parallel to, prior to, or following additional methods of treatment. For example, CD25 DNA vaccines may be used in combination with T cell vaccination, or in combination with vaccination with a target antigen of the disease being treated (see, for example, Cohen et al., 2004 and references cited therein).

The following examples are presented in order to more fully illustrate some embodiments of the invention. They should, in no way be construed, however, as limiting the broad scope of the invention.

### EXAMPLES

### Materials and Methods

Plasmids and DNA Vaccination

The coding sequence for the α-chain of the rat IL-2 receptor (CD25; SEQ ID NO:10, gi:204911) was cloned into the pcDNA3 expression vector (Invitrogen) in the BamHI-XbaI sites; the mouse γ-chain (SEQ ID NO:11, gi:31982446) was cloned in the BamHI-XhoI sites. The empty pcDNA3 vector was used as a control. Plasmid DNA was prepared in large scale using the Qiagen Plasmid Maxi Kit (Qiagen, Hilden, Germany). DNA was eluted to a final concentration of 1 mg/ml. Groups of 8 rats were injected intramuscularly to the quadriceps with 100 μl/rat of 10 μM cardiotoxin (Sigma, St. Louis, Mo.) to increase the efficiency of DNA uptake (Danko et al., 1994). Three vaccinations were given at 10-day intervals, beginning 5 days after cardiotoxin injection, 100 μg/rat of DNA in the same site.

Serum Antibodies Induced by DNA Vaccination

Sera of DNA-vaccinated rats were obtained 10 days after the 3^{rd} DNA vaccination and assayed for IgG antibodies to peptides of CD25, CD122, TNFR1 and a control peptide p53-1 (see sequences below). NUNC-Maxisorp plates (NUNC, Roskilde, Denmark) were coated over-night at 4° c. with 20 μg/ml of test peptides, blocked with 1% BSA for 2 hours at room-temperature, and a 1:15 dilution of sera was added for incubation over-night at 4° c. Mouse anti-rat IgG alkaline-phosphatase (AP) conjugated 2^{nd} antibody was added in a 1:1000 dilution for 1 hour at room-temperature, AP substrate was added and the plates were read at O.D. 405.

AA Induction and Scoring

Heat killed Mycobacterium tuberculosis (Mt) strain H37Ra (Difco, Detroit, Mich.) was finely ground using a pestle and mortar, and suspended to a final concentration of 10 mg/ml in IFA. To induce AA, female Lewis rats were injected at the base of the tail with 100 μl of the Mt suspension containing 1 mg of Mt. AA was scored by direct observation of the four limbs in each individual. A relative score between 0 and 4 was assigned to each limb, based on the degree of joint inflammation, redness and deformity. The maximum possible score for a subject rat was 16. AA was also quantified by measuring the hind limb ankle diameter with a caliper on day 26. The disease reaches its peak severity between days 22 and 26.

T Cell Clones

For ergotypic stimulation, the Lewis rat A6 T cell clone was used, specific for myelin basic protein (MBP; Mor et al., 1996b). Activated A6 T cells can mediate EAE but not AA when administered live to Lewis rats. A6 stimulation medium was composed of DMEM supplemented with 2-ME (5×10⁻⁵M), L-glutamine (2 mM), sodium pyruvate (1 mM), penicillin (100 U/ml), streptomycin (100 μg/ml), nonessential amino acids (1 ml/100 ml), 1% autologous serum and 10 μg/ml of the specific antigen—guinea pig MBP. After 3 days of stimulation, A6 cells were transferred to rest medium, as above but without MBP, and containing 10% FCS instead of autologous rat serum and 10% TCGF (T cell growth factors prepared from the supernatant of ConA activated spleen cells; Gillis et al., 1978). Activated A6 cells (A6-S) were used on day 3 of their stimulation, and resting A6 cells (A6-R) were used on day 7 of their rest cycle.

Peptides

Peptides were synthesized using the F-moc technique with an automatic multiple peptide synthesizer (AMS 422, ABIMED, Langenfeld, Germany). The purity of the peptides was analyzed by HPLC and amino acid composition. Peptide antigens used in proliferation experiments were two IL-2R α-chain (a1, a2), two β-chain (b1, b2) peptides, and a TNFR1 peptide described elsewhere (Mor et al., 1996). The sequences are: a1—TTDTQKSTQSVYQENLAGHCR (SEQ ID NO:3); a2—ASEESQGSRNSFPESEACPT (SEQ ID NO:4); b1—IFLETLTPDTSYELQVRVIA (SEQ ID NO:5); b2—SVDLLSLSVVCWEEKGWRRV (SEQ ID NO:6); TNFR1-WKEFMRLLGLSEHEIERLEL (SEQ ID NO:7). The control peptide p53-1 is composed of the first 20 amino acids of the p53 protein—MTAMEESQSDISLELPLSQE (SEQ ID NO:8). Target antigens associated with AA were the p180 peptide composed of amino acids 176-190 of Mt HSP65: EESNTFGLQLELTEG (SEQ ID NO:9), and the purified protein derivative (PPD) of Mt (Statens Seruminstitut, Denmark).

T-Cell Proliferation Assay

Draining lymph node (DLN) cells (inguinal and popliteal) were pooled from three rats of each experimental group and cultured in quadruplicates, 2×10⁵/200 μl in round bottom microtiter wells (NUNC, Roskilde, Denmark). Peptides or PPD antigen was used at a final concentration of 20 μg/ml, and ConA was used at a concentration of 1.25 g/ml as a positive control for T cell proliferation. In the T cell coculture proliferations, A6 cells were irradiated (5000 R) and added to the test cultures in 2-fold dilutions, starting from 5×10⁴ cells per well. Stimulation medium was composed of DMEM supplemented with 2-ME (5×10⁻⁵M), L-glutamine (2 mM), sodium pyruvate (1 mM), penicillin (100 U/ml), streptomycin (100 μg/ml), nonessential amino acids (1 ml/100 ml) and 1% autologous serum. Cultures were incubated for 72 hours at 37° C. in humidified air containing 7% CO2. Each well was pulsed with 1 μCi of [³H]Thymidine (Amersham, Buckinghamshire, UK) for the last 16 hours. The cultures were then harvested and cpm were determined using a beta counter. The stimulation index (SI) was calculated as the ratio of the mean cpm for each quadruplicate (containing the test antigen) to the mean cpm of spontaneous proliferation (wells containing LN cells without antigen).

Cytokine Assays

Supernatants from the T cell proliferation experiments were collected at 72 hours. Rat IFNγ, TNFα, IL-10 and IL-4 were quantified by ELISA using Pharmingen's OPTEIA™ kits for each of the cytokines (Pharmingen, San-Diego, Calif.), following the manufacturer's protocols. Rat TGFβ1 was quantified using the TGFβ1 Eₘₐₓ® ImmunoAssay System (Promega, Madison, Wis.) according to the manufacturer's instructions.

Statistical Significance

The InStat 2.01 software was used for statistical analysis. Student t test and the Mann-Whitney test were carried out to assay the differences between experimental groups.

### Example 1

### Anti-Ergotypic T Cells are Present in Naïve Rats and are Down-Regulated Upon AA Induction

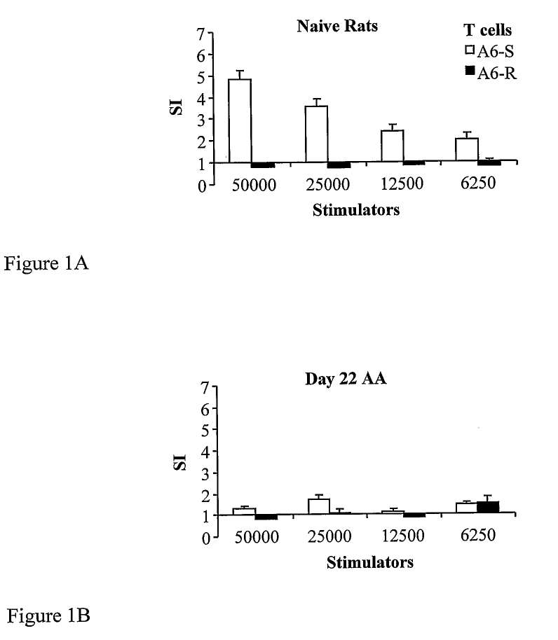

It was first tested whether there existed a basal anti-ergotypic activity, and whether it might be affected by the induction of AA, an autoimmune disease associated with T cell activation (Wauben et al., 1994). Naïve LN cells collected from inguinal and popliteal lymph nodes were cocultured with irradiated A6 clone cells, either activated or resting, to test their anti-ergotypic response. FIG. 1A shows that naïve LN cells exhibit a natural anti-ergotypic response to activated T cells (A6-S), but not to resting (A6-R) cells. The same experiment was then done with DLN cells taken from rats after AA induction. As can be seen in FIG. 1B, the induction of the disease was associated with down-regulation of the natural anti-ergotypic response, reaching its lowest levels at the peak of the disease.

### Example 2

### DNA Vaccination with CD25 Protects from Adjuvant Arthritis

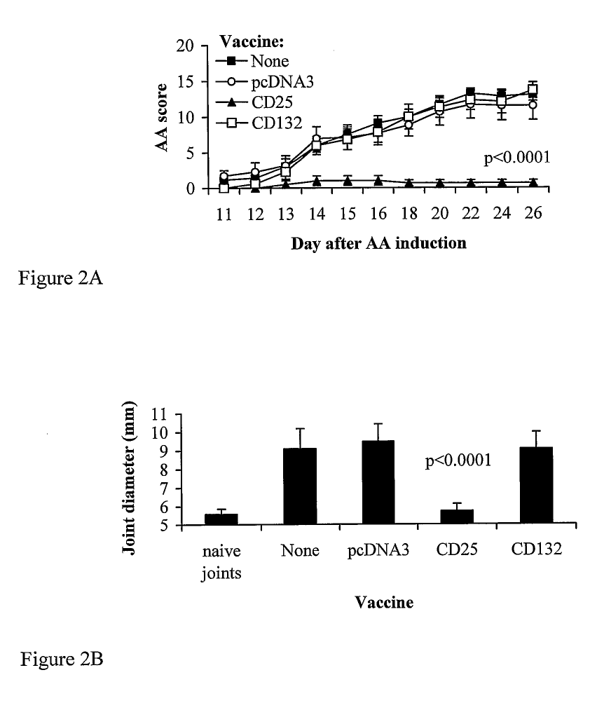

To study anti-ergotypic vaccination, Lewis rats were vaccinated with DNA encoding the IL-2R α-chain (CD25), which is up-regulated on activated T cells (Taniguchi and Minami, 1993; Minami et al., 1993), and which was found to serve as an ergotope (Mor et al., 1996). As controls, we vaccinated rats with the empty vector (pcDNA3) or with DNA encoding the constitutively expressed IL-2R γ-chain (CD132). FIG. 2A shows that rats vaccinated with the pcDNA3 empty vector or with the CD132 DNA developed the same level of disease as did the control rats. In contrast, rats vaccinated with the CD25 gene were protected from AA. Protection was evident also by comparing the degree of ankle swelling, as shown in FIG. 2B. Thus, DNA vaccination with a single ergotope was effective in protecting rats from AA.

### Example 3

### Vaccination with CD25 DNA Induces IgG Antibodies to CD25 Peptides

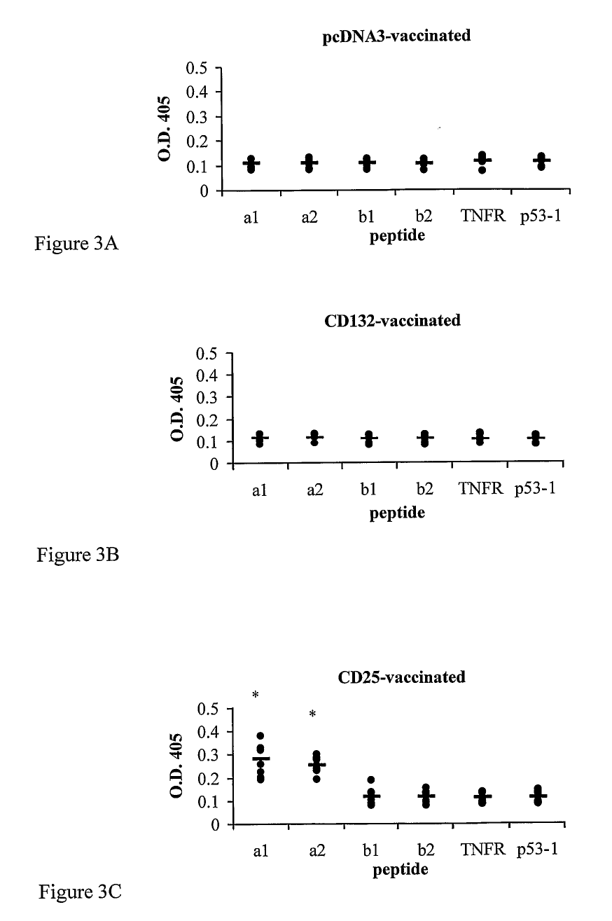

To investigate whether DNA vaccination with CD25 might induce specific antibodies, rats were vaccinated with CD25 DNA. Control groups were vaccinated with CD132 DNA, with an empty vector DNA or not vaccinated. Ten days after the 3^{rd} DNA vaccine, sera were obtained and tested for antibodies to five ergotope peptides: two peptides of the IL-2R α-chain, two of the β-chain and a peptide of TNFR1. All five peptides had been found to be immunogenic in the Lewis rat (Mor et al., 1996). As shown in FIG. 3, the CD25 DNA vaccine induced a low but significant specific IgG response to the two CD25 peptides and not to peptides of the other ergotopes. CD132 DNA or an empty vector vaccine did not induce IgG responses to any of the peptides.

### Example 4

### Vaccination with CD25 DNA Followed by AA Induction Induces T Cell Proliferation to Peptides of CD25 and CD122

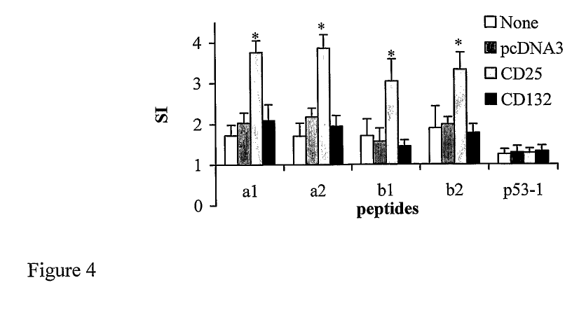

Although CD25 DNA vaccination induced IgG antibodies to CD25 peptides (FIG. 3), proliferative T-cell responses to CD25 peptides in the absence of AA were not detected (not shown). Since AA induction is followed by the activation of autoreactive effector T cells, it was tested whether induction of AA might be associated with an enhanced anti-ergotypic response induced by DNA vaccination. AA was induced in vaccinated rats and DLN were obtained on day 22 after disease induction. As shown in FIG. 4, DLN cells taken from the three control groups showed only background levels of proliferation to the α-chain and β-chain peptides of CD25. However, DLN cells from the group vaccinated with the CD25 gene exhibited a significant response to the α-chain peptides and, surprisingly, also to the β-chain peptides (FIG. 4). No proliferation to the non-related p53 control peptide was observed. Thus, induction of a proliferative response to peptides of both the CD25 and CD122 chains followed specific DNA vaccination with the CD25 gene and the induction of AA.

### Example 5

### Protection from AA is Associated with Preservation of the Anti-Ergotypic Proliferative Response

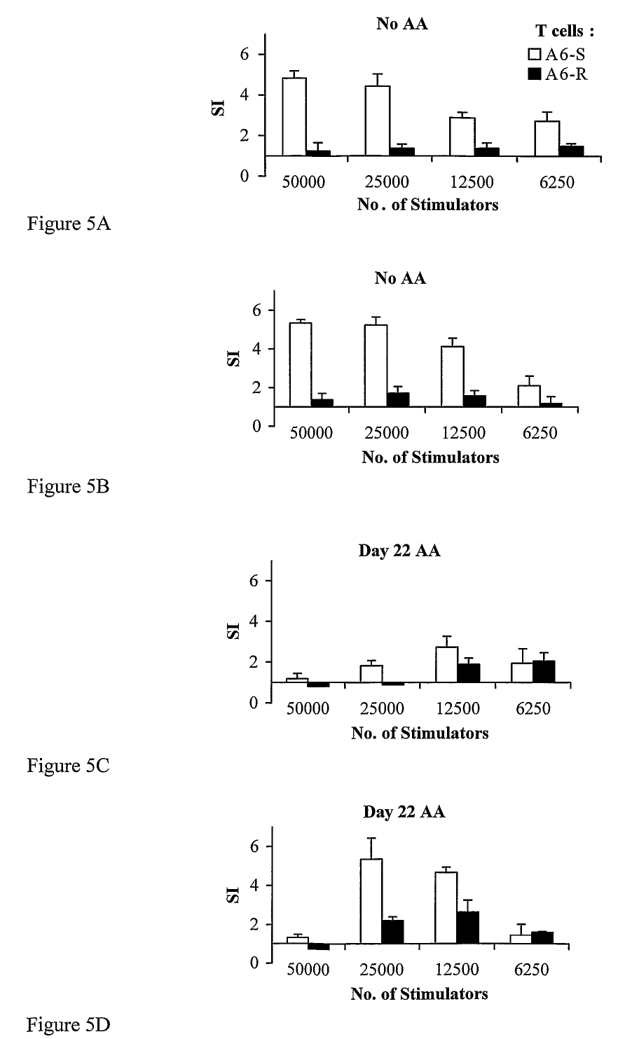

To relate the mechanism of protection by CD25 vaccination to the induction of the anti-ergotypic response, DLN cells from the rats were studied for their ability to proliferate in response to activated or resting syngeneic T cells. Two different time points were studied after DNA vaccination with CD25 or pcDNA3: before AA induction (FIG. 5A-B) or at day 22 after AA induction (FIG. 5C-D). As stimulators, the A6 T cell clone, activated (A6-S) or resting (A6-R) were used (Mor et al., 1996b). As can be seen in FIGS. 5A-B, before AA induction neither the CD25 (5B) nor the pcDNA3 (5A) DNA vaccines could amplify the natural anti-ergotypic response found in naïve rats (compare FIGS. 5A,B with FIG. 1A). The difference between the groups was found only after the induction of AA: In the group that had been vaccinated with the pcDNA3 vector, a significant decrease in the anti-ergotypic proliferative response was observed (FIG. 5C), like that seen in naïve rats undergoing AA. But the CD25 DNA vaccinated group retained their anti-ergotypic proliferative response to activated A6 T cells (FIG. 5D). Only a weak response was seen to the resting A6 T cells. Thus, effective DNA vaccination with the CD25 gene prevented the decline of the natural anti-ergotypic proliferation response that otherwise accompanies AA.

### Example 6

### Cytokine Profile of Anti-Ergotypic T Cells

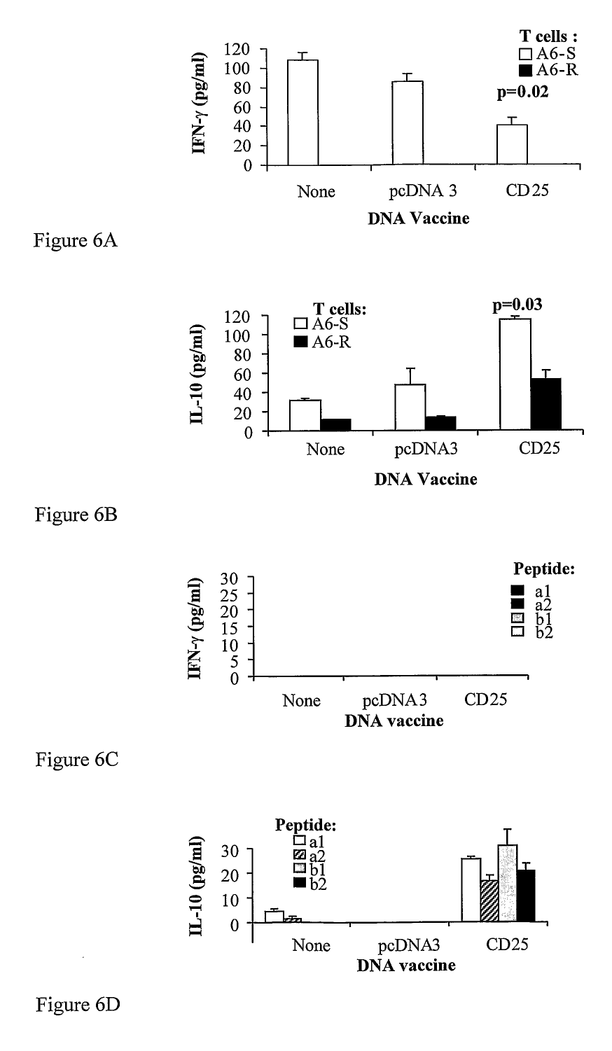

To document the cytokine profile secreted by anti-ergotypic T cells obtained from immunized rats, their DLN cells were stimulated by activated or resting A6 T cells or α or β peptides on day 22 of AA, and culture media were analyzed for the presence of IFNγ and IL-10. As can be seen in FIG. 6, DLN cells from both non-protected groups (non-treated and pcDNA3 vaccinated) did secrete IFNγ and some IL-10 although they did not proliferate in response to activated T cells (FIG. 6A). In contrast, DLN cells from protected rats immunized with CD25 DNA proliferated to activated T cells and secreted significantly increased amounts of IL-10 and less IFNγ (FIG. 6A-B).

DLN cells proliferating to the α or β peptides taken from the CD25 protected rats only, secreted IL-10 and did not secret IFNγ. DLN cells from the two non-protected groups secreted neither IFNγ nor IL-10 (FIG. 6C-D).

### Example 7

### Effect of DNA Vaccination on T Cell Proliferation to AA Antigens

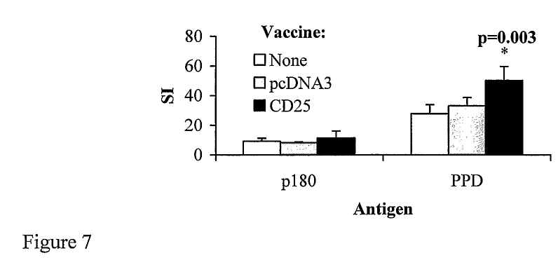

Protective CD25 vaccination to modify T-cell immunity to antigens associated with AA, namely the p180 peptide (amino acids 176-190 of the Mycobacterial HSP65) and PPD (the whole purified protein derivative of Mt) was also tested. Peptide p180 was found to be the target of arthritogenic T cells in AA (van Eden et al., 1988) and PPD contains a mixture of Mycobacterial antigens. Twenty-two days after AA induction, DLN cells were taken from the three groups and stimulated in vitro using either of the two antigens. As can be seen in FIG. 7, there was no significant difference between the three groups in their T cell proliferation to the p180 peptide. However, T cell proliferation to PPD was significantly higher in the protected rats vaccinated with the CD25 gene.

### Example 8

### Protection is Associated with a Cytokine Shift from a Th1-Like to a Th2-Like Phenotype

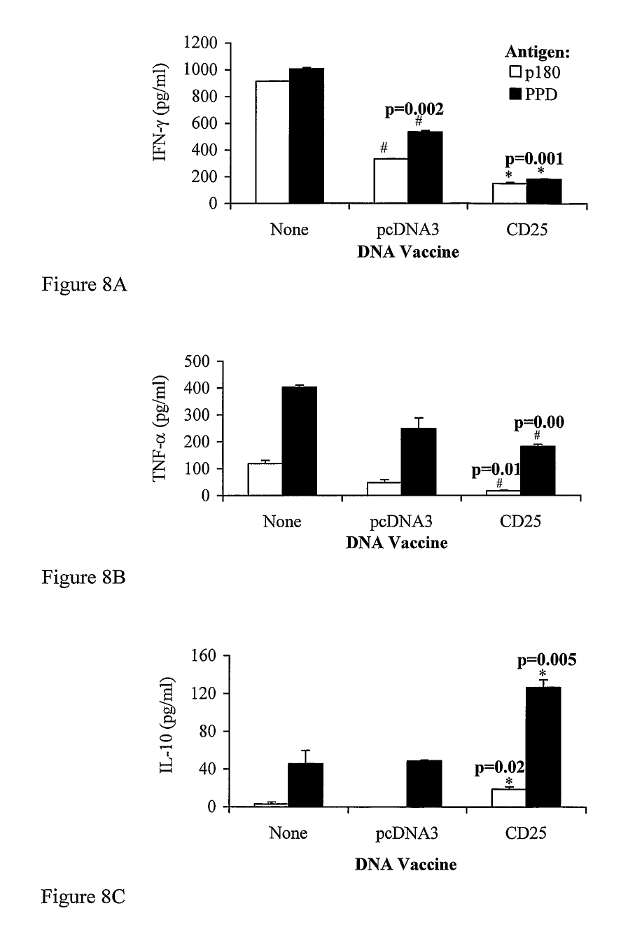

The effect of CD25 DNA vaccination on the cytokine profile was analyzed using media taken from the proliferating T cells described above was analyzed. FIG. 8 shows the results: DLN cells from the untreated control AA rats secreted high levels of IFNγ (FIG. 8A) and TNFα (FIG. 8B) in response to stimulation with the p180 peptide or with PPD. DLN cells from animals vaccinated with the empty pcDNA3 vector, although not protected, secreted less IFNγ. Note however that there was a significant decrease in IFNγ and TNFα secretion by cells taken from the CD25 protected rats. The opposite pattern was detected when the same cells were tested for the secretion of IL-10, a Th2 cytokine. While both control groups secreted low levels of IL-10 in response to PPD or p180, the CD25 DNA vaccinated group, protected from the disease, exhibited a significant increase in IL-10 secretion (FIG. 8C). Secretion of IL-4 and TGFβ was not detectable in these samples.

Anti-ergotypic regulation stimulated by CD25 DNA vaccination can thus down-regulate AA. The cytokine balance between the anti-ergotypic T cells and the AA-associated T cells may affect the whole cytokine environment. In non-vaccinated rats, the arthritogenic T cells causing the disease seem to be the ones controlling the cytokine environment by secreting mainly Th1 cytokines, IFNγ and TNFα. These Th1 cytokines might also have an inhibitory effect on the activation of the anti-ergotypic T cells, which do not proliferate but secrete IFNγ. In contrast, CD25 DNA vaccination could boost the anti-ergotypic T cells, leading to their preservation and their secretion of IL-10. The IL-10 could help drive the differentiation of the otherwise pathogenic T cells towards a Th2 phenotype.

The results presented in these Examples are summarized in Table I herein:

TABLE I
Summary of findings.

| | | DLN cell proliferation to | | | | | | | |
|---|---|---|---|---|---|---|---|---|---|
| | | Whole | Whole | | | | Cytokine secretion to | | |
| DNA | AA | activated | resting | α/β | Control | AA | Whole | α/β | AA |
| vaccine | induction | T cells | T cells | peptides | peptide | antigens | T cells | peptides | antigens |
| None | − | + | − | − | − | − | − | − | − |
| | + | − | − | − | − | + | Th1 * | − | Th1 |
| pcDNA3 | − | + | − | − | − | − | − | − | − |
| | + | − | − | − | − | + | Th1 | − | Th1 |
| | − | + | − | − | − | − | − | − | − |
| CD25 | + | + | −/+ | + | − | ++ | Th2 ^{#} | Th2 | Th2 |
| | (protected) | | | | | | | | |

* Th1 indicates relatively high IFNγ and low IL-10.
^{#} Th2 indicates relatively high IL-10 and low IFNγ.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### REFERENCES

1. Lohse, A. W., F. Mor, N. Karin, and I. R. Cohen. 1989. Science 244:820.
2. Mor, F., B. Reizis, I. R. Cohen, and L. Steinman. 1996. J Immunol 157:4855.
3. Taniguchi, T., and Y. Minami. 1993. Cell 73:5.
4. Minami, Y., T. Kono, T. Miyazaki, and T. Taniguchi. 1993. Annu Rev Immunol 11:245.
5. Danko, I., J. D. Fritz, S. Jiao, K. Hogan, J. S. Latendresse, and J. A. Wolff. 1994. Gene Ther 1:114.
6. Mor, F., M. Kantorowitz, and I. R. Cohen. 1996(b). J Neurosci Res 45:670.
7. Gillis, S., M. M. Ferm, W. Ou, and K. A. Smith. 1978. J Immunol 120:2027.
8. Wauben, M. H. M., J. P. A. Wagenaar-Hilbers, and W. van-Eden. 1994. Adjuvant Arthritis. In Autoimmune Disease Models: A Guidebook. I. R. Cohen, and A. Miller, Eds. Academic Press, Inc.
9. van Eden, W., J. E. Thole, R. van der Zee, A. Noordzij, J. D. van Embden, E. J. Hensen, and I. R. Cohen. 1988. Nature 331:171.
10. Cohen, I. R. 2001. Vaccine 20:706.
11. Kumar, V., J. Maglione, J. Thatte, B. Pederson, E. Sercarz, and E. S. Ward. 2001. Int Immunol 13:835.
12. Sambrook et al., Molecular Cloning: A Laboratory Manual, Cold Spring Harbor Labs Press, 1989.
13. Wolff et al., 1990, Science 247, 1465-1468.
14. Stribling et al., Proc. Natl. Acad. Sci. USA 189:11277-11281, 1992.
15. Stewart, J. M. and Young, J. D. (1963), “Solid Phase Peptide Synthesis,” W. H. Freeman Co. (San Francisco).
16. Meienhofer, J (1973). “Hormonal Proteins and Peptides,” vol. 2, p. 46, Academic Press (New York).
17. Schroder, G. and Lupke, K. (1965). The Peptides, vol. 1, Academic Press (New York).
18. Reizis, B., F. Mor, M. Eisenstein, H. Schild, S. Stefanovic, H. G. Rammensee, and I. R. Cohen. 1996. Int Immunol 8:1825.
19. Cohen, I. R., Quintana, F. J. and A. Mimran, 2004, J Clin Invest. 114(9):1227-32.

1. A DNA vaccine composition comprising a recombinant construct comprising an isolated nucleic acid sequence encoding an antigen selected from CD25, homologs and fragments thereof; the nucleic acid sequence being operably linked to one or more transcription control sequences; and a pharmaceutically acceptable carrier, adjuvant, excipient or diluent.

2. The composition of claim 1, wherein the CD25 is human CD25.

3. The composition of claim 1, wherein the isolated nucleic acid sequence has a nucleic acid sequence as set forth in SEQ ID NO:1.

4. The composition of claim 1, wherein the isolated nucleic acid sequence encodes an antigen having an amino acid sequence as set forth in any one of SEQ ID NOS:2-4.

5. The composition of claim 1, wherein the composition is a naked DNA vaccine.

6. The composition of claim 1, wherein said carrier is selected from the group consisting of liposomes, micelles, emulsions and cells.

7. The composition of claim 1, wherein said transcription control sequences are selected from the group consisting of: RSV control sequences, CMV control sequences, retroviral LTR sequences, SV-40 control sequences and β-actin control sequences.

8. The composition of claim 1, wherein said recombinant construct is a eukaryotic expression vector.

9. The composition of claim 8, wherein said eukaryotic expression vector is selected from the group consisting of: pcDNA3, pcDNA3.1(+/−), pZeoSV2(+/−), pSecTag2, pDisplay, pEF/myc/cyto, pCMV/myc/cyto, pCR3.1, pCI, pBK-RSV, pBK-CMV and pTRES.

10. A method of preventing or inhibiting the development of a T-cell mediated pathology, comprising administering to a subject in need thereof a therapeutically effective amount of a pharmaceutical composition comprising: (a) a recombinant construct, said recombinant construct comprising an isolated nucleic acid sequence encoding an antigen selected from: CD25, homologs and fragments thereof, wherein the nucleic acid sequence is operably linked to one or more transcription control sequences; and (b) a pharmaceutically acceptable carrier, excipient or diluent.

11. The method of claim 10, wherein the CD25 is human CD25.

12. The method of claim 10, wherein the isolated nucleic acid sequence is as set forth in SEQ ID NO:1.

13. The method of claim 10, wherein the isolated nucleic acid sequence encodes an antigen having an amino acid sequence as set forth in any one of SEQ ID NOS:2-4.

14. The method of claim 10, wherein said T cell-mediated pathology is an autoimmune disease.

15. The method of claim 14, wherein said T cell-mediated autoimmune disease is selected from the group consisting of: multiple sclerosis, rheumatoid arthritis, autoimmune neuritis, systemic lupus erythematosus, psoriasis, Type I diabetes mellitus, Sjogren's disease, thyroid disease and myasthenia gravis.

16. The method of claim 10, wherein said T cell-mediated pathology is graft rejection.

17. The method of claim 10, wherein said T cell-mediated pathology is a Th1-mediated inflammatory disease.

18. The method of claim 10, wherein the antigen is expressed in sufficient amount and duration to increase anti-ergotypic T cell response in said subject, thereby inhibiting or preventing the development of said T-cell mediated pathology.

19. The method of claim 18, wherein said increase in anti-ergotypic T cell response is characterized by a reduction in the secretion of IFNγ and an increase in the secretion of IL-10.

20. The method of claim 10, wherein the nucleic acid composition is administered as naked DNA.

21. The method of claim 10, wherein said subject is human.

22. A method for preventing or inhibiting the development of a T-cell mediated pathology comprising the steps of (a) obtaining cells from a subject; (b) transfecting the cells in vitro with a recombinant construct comprising an isolated nucleic acid sequence encoding an antigen selected from: CD25, homologs and fragments thereof, the nucleic acid sequence being operably linked to one or more transcription control sequences; and (c) reintroducing a therapeutically effective number of the transfected cells to the subject, thereby preventing or inhibiting the development of the T-cell mediated pathology.

23. The method of claim 22, wherein the CD25 is human CD25.

24. The method of claim 22, wherein the isolated nucleic acid sequence is as set forth in SEQ ID NO:1.

25. The method of claim 22, wherein the isolated nucleic acid sequence encodes an antigen having an amino acid sequence as set forth in any one of SEQ ID NOS:2-4.

26. The method of claim 22, wherein said T cell-mediated pathology is an autoimmune disease.

27. The method of claim 26, wherein said T cell-mediated autoimmune disease is selected from the group consisting of: multiple sclerosis, rheumatoid arthritis, autoimmune neuritis, systemic lupus erythematosus, psoriasis, Type I diabetes mellitus, Sjogren's disease, thyroid disease and myasthenia gravis.

28. The method of claim 22, wherein said T cell-mediated pathology is graft rejection.

29. The method of claim 22, wherein said T cell-mediated pathology is a Th1-mediated inflammatory disease.

30. The method of claim 22, wherein the antigen is expressed in sufficient amount and duration to increase anti-ergotypic T cell response in said subject, thereby inhibiting or preventing the development of said T-cell mediated pathology.

31. The method of claim 30, wherein said increase in anti-ergotypic T cell response is characterized by a reduction in the secretion of IFNγ and an increase in the secretion of IL-10.

32. The method of claim 22, wherein said subject is human.

33-48. (canceled)
